(19) United States
(12) Patent Application Publication
(10) Pub. No.: US 2019/0074083 A1
(43) Pub. Date: Mar. 7, 2019

(54) METHOD AND DATA PROCESSING UNIT FOR DETERMINING CLASSIFICATION DATA FOR ADAPTION OF AN EXAMINATION PROTOCOL

(71) Applicant: Siemens Healthcare GmbH, Erlangen (DE)

(72) Inventors: Dorothee JUNG, Erlangen (DE); Ute FEUERLEIN, Erlangen (DE); Rainer RAUPACH, Heroldsbach (DE); Thomas ALLMENDINGER, Forchheim (DE); Christiane KOCH, Eggolsheim (DE)

(73) Assignee: Siemens Healthcare GmbH, Erlangen (DE)

(21) Appl. No.: 16/118,660

(22) Filed: Aug. 31, 2018

(30) Foreign Application Priority Data
Sep. 7, 2017 (DE) 102017215829.8

(51) Int. Cl. G16H 30/20; G06F 17/30; G06N 99/00; G06K 9/62

(52) U.S. Cl. CPC G16H 30/20; G06F 17/3028; G06K 9/6253; G06K 9/6282; G06K 9/6262; G06N 99/005

(57) ABSTRACT

A method is for determining classification data for adaption of an examination protocol based on a basic examination protocol of a medical imaging examination as a function of status parameters of the medical imaging examination. In an embodiment, the method includes supplying a set of training data sets, every training data set in each case including a status parameter data set with values of the status parameters of the medical imaging examination and an item of adaption information associated with the status parameter data set. The adaption information relates to an adaption of the examination protocol based on the basic examination protocol of the medical imaging examination, in particular as a function of the status parameters. Finally, the method includes determining the classification data based on a machine learning algorithm and the set of training data sets.

### PRIORITY STATEMENT

The present application hereby claims priority under 35 U.S.C. § 119 to German patent application number DE102017215829.8 filed Sep. 7, 2017, the entire contents of which are hereby incorporated herein by reference.

### FIELD

Embodiments of the invention relate to a method and to a data processing unit for determining classification data, to a use of classification data for optimizing an examination protocol adaption algorithm, to a use of classification data for optimizing a basic examination protocol database, to a method and to a data processing unit for adaption of an examination protocol, to a computer program product and to a computer-readable medium.

### BACKGROUND

As a rule, basic examination protocols for the most common examinations are permanently stored on imaging systems. However, these are not just used in this rigid form, but are changed ad hoc as a function of specific situations, for example the condition of the patient, in particular their laboratory values, heart rate, habitus, etc. These can relate for example to acquisition parameters, reconstruction parameters and/or contrast medium parameters.

Furthermore, an adapted calculation basis can be supplied in this way for algorithms, in particular reconstruction algorithms and/or image processing algorithms. To keep the number of basic examination protocols manageable, not all sub-types are stored as a separate protocol. A procedure of this kind requires additional communication of rules and is prone to faults.

At the same time, many users do not know that there are automation mechanisms on the imaging systems which enable automatic adaption of examination protocols as a function of status parameters. This can in particular be an automatic dosing system, for example as a function of the attenuation of the X-ray radiation through the patient, automatic determination of the optimum cardiac phase for acquisition and reconstruction, for example as a function of the heart rate and/or heart rate variability or the like. In particular, this relates to functionalities which are newly added, for example due to software upgrades, and have to be incorporated in the existing protocols with expense and an imperative understanding of the function.

Nowadays, as a rule, there are specifications supplementary to the basic examination protocols, which relate to manual adaption of examination protocols starting from the basic examination protocol as a function of the status parameters. These are available to the user as an electronic document on a separate computer, in printed form or sometimes even just as a collection of handwritten notes. With regard to possibilities for automation of the medical imaging device that is individual to the patient, training is conventionally provided by the manufacturer but is often not fully understood by the user. As a consequence, automatisms that are individual to the patient are not used at all in many cases or are even incorrectly used.

The manual adaption of the examination protocols is typically based on the data of an optimum reference patient. However, in many case this reference data is non-configurable and, apart from a few exceptions, such as, for example a weight specification, does not represent any further properties of a real patient, such as, for example a heart rate, pre-existing conditions or the like. A user therefore often cannot optimally classify the patient to be examined using the reference patient, in particular in the case of a patient-specific adaption.

Furthermore, until now it has not been possible to comprehensively check the plausibility of input threshold values and reference points in the default value since the input data typically only takes effect in a real scanning process. Transparency and understanding suffer due to this separation of the input of the data and its consequences which these have in the scanning process. In turn this can lead to the basic examination protocols scarcely being checked, understood or changed and to the potential for optimization, in particular in the case of automatic adaption individual to the patient, often not being exploited.

U.S. Pat. No. 8,000,510 B2 discloses a method for controlling a sectional image acquisition system in which a scanning protocol is selected from a number of scanning protocols.

U.S. Pat. No. 8,401,872 B2 discloses a method for operating a medical diagnostic device with the aid of which medical issues are to be addressed.

U.S. Pat. No. 8,687,762 B2 discloses a CT system for scanning a patient, having at least one computer system, which can control the CT system, with an evaluation unit for a specified logical decision tree being integrated in the computer system.

U.S. Pat. No. 9,615,804 B2 discloses a method for image generation and image evaluation in the medical sector, wherein via a specified medical modality, in particular a computer tomograph, raw data is generated as a function of specified modality parameters.

U.S. Pat. No. 9,636,077 B2 discloses a method for automatic selection of a scanning protocol for tomographic acquisition of an X-ray image of a patient.

### SUMMARY

At least one embodiment of the invention enables improved adaption of an examination protocol based on a basic examination protocol as a function of status parameters of the medical imaging examination. Further advantageous embodiments of the invention are considered in the claims.

At least one embodiment of the invention relates to a method for determining classification data for adaption of an examination protocol based on a basic examination protocol of a medical imaging examination as a function of status parameters of the medical imaging examination, wherein the method comprises:

supplying a set of training data sets, wherein every training data set in each case has a status parameter data set with values of the status parameters of the medical imaging examination and an item of adaption information associated with the status parameter data set, wherein the adaption information relates to an in particular manual adaption of the examination protocol, for example by one or more user(s), based on the basic examination protocol of the medical imaging examination, in particular as a function of the status parameters; and

determining the classification data based on a machine learning algorithm and the set of training data sets.

At least one embodiment of the invention also relates to a data processing unit for determining classification data for an adaption of an examination protocol based on a basic examination protocol of a medical imaging examination as a function of status parameters of the medical imaging examination, comprising:

a training data set supply unit designed for supplying a set of training data sets, wherein every training data set in each case has a status parameter data set with values of the status parameters of the medical imaging examination and an item of adaption information associated with the status parameter data set, wherein the adaption information relates to an in particular manual adaption of the examination protocol, for example by one or more user(s), based on the basic examination protocol of the medical imaging examination, in particular as a function of the status parameters; and

a classification data determining unit designed for determining the classification data based on a machine learning algorithm and the set of training data sets.

At least one embodiment of the invention also relates to a method of using classification data, which has been determined according to a method for determining classification data according to one or more of the embodiment(s) disclosed in this application, for optimizing an examination protocol adaption algorithm, which is designed in particular for automatic adaption of an examination protocol based on a basic examination protocol of a medical imaging examination as a function of status parameters of the medical imaging examination. Optimizing an examination protocol adaption algorithm can in particular be taken to mean training of the examination protocol adaption algorithm.

At least one embodiment of the invention also relates to a method of using classification data, which has been determined according to a method for determining classification data according to one or more of the embodiment(s) disclosed in this application, for optimizing a basic examination protocol database which has a plurality of basic examination protocols,

wherein based on the classification data, at least one further basic examination protocol for a scan is determined in the basic examination protocol database and/or

wherein the basic examination protocols of the plurality of basic examination protocols are classified based on the classification data.

At least one embodiment of the invention also relates to a method for adaption of an examination protocol of a medical imaging examination, wherein the method comprises:

selecting a basic examination protocol of the medical imaging examination;

supplying a data structure in which values of status parameters of a status parameter data set can be stored and changed; and

adaption of an examination protocol based on the basic examination protocol as a function of the status parameters of the status parameter data set.

At least one embodiment of the invention also relates to a data processing unit for adaption of an examination protocol of a medical imaging examination, wherein the data processing unit comprises:

selection unit designed for selecting a basic examination protocol of the medical imaging examination;

data structure supply unit designed for supplying a data structure in which values of status parameters of a status parameter data set can be stored and changed; and

adaption unit designed for adaption of an examination protocol based on the basic examination protocol as a function of the status parameters of the status parameter data set.

At least one embodiment of the invention also relates to a method for optimizing a basic examination protocol database, including a plurality of basic examination protocols, the method comprising:

determining the classification data based upon the method of an embodiment of the application; and at least one of
determining, based on the classification data determined, at least one further basic examination protocol for a scan in the basic examination protocol database, and
classifying the basic examination protocols of the plurality of basic examination protocols based on the classification data determined.

At least one embodiment of the invention also relates to a method for adaption of an examination protocol of a medical imaging examination, the method comprising:

selecting a basic examination protocol of the medical imaging examination;

supplying a data structure, to store changeable values of status parameters of a status parameter data set; and

adapting the examination protocol based on the basic examination protocol selected as a function of the status parameters of the status parameter data set.

At least one embodiment of the invention also relates to a method for adaption of an examination protocol of a medical imaging examination, the method comprising:

selecting a basic examination protocol of the medical imaging examination;

supplying a data structure, to store changeable values of status parameters of a status parameter data set; and

adapting the examination protocol based on the basic examination protocol selected as a function of the status parameters of the status parameter data set,

wherein the adapting of the examination protocol takes place using an examination protocol adaption algorithm, optimized using classification data determined according to the method of an embodiment of the application.

At least one embodiment of the invention also relates to a non-transitory computer program product having a computer program, which can be loaded directly into a storage device of a computer, having program segments in order to carry out all steps of a method according to one or more of the embodiment(s) disclosed in this application when the computer program is executed in the computer.

At least one embodiment of the invention also relates to a non-transitory computer-readable medium on which program segments that can be read and executed by a computer are stored in order to carry out all steps of a method according to one of the embodiment(s) disclosed in this application when the program segments are executed by the computer.

### BRIEF DESCRIPTION OF THE DRAWINGS

In the drawings:

FIG. 1 shows a flowchart of a method for determining classification data,

FIG. 2 shows a schematic illustration of a data processing unit for determining classification data,

FIG. 3-5 shows a schematic illustration of the classification data for adaption of an examination protocol,

FIG. 6 shows a flowchart of a method for adaption of an examination protocol,

FIG. 7 shows a schematic illustration of a data processing unit for adaption of an examination protocol,

FIG. 8 shows a user interface for adaption of an examination protocol, and

FIG. 9 shows a medical imaging device.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. The present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “exemplary” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

At least one embodiment of the invention relates to a method for determining classification data for adaption of an examination protocol based on a basic examination protocol of a medical imaging examination as a function of status parameters of the medical imaging examination, wherein the method comprises:

supplying a set of training data sets, wherein every training data set in each case has a status parameter data set with values of the status parameters of the medical imaging examination and an item of adaption information associated with the status parameter data set, wherein the adaption information relates to an in particular manual adaption of the examination protocol, for example by one or more user(s), based on the basic examination protocol of the medical imaging examination, in particular as a function of the status parameters; and

determining the classification data based on a machine learning algorithm and the set of training data sets.

One embodiment of the invention provides that the classification data forms a decision tree and/or that the machine learning algorithm is based on recursive partitioning. In particular, the classification data can be determined by training the decision tree by way of recursive partitioning. Based on the decision tree, for example at least one examination protocol parameter of the examination protocol can be defined and/or modified starting from the basic examination protocol and as a function of the status parameters.

In the context of at least one embodiment of this application, a machine learning algorithm is in particular taken to mean an algorithm which is designed for machine learning. A machine learning algorithm can be implemented for example with the aid of decision trees, mathematical functions and/or general programming languages. The machine learning algorithm can be designed for example for monitored learning and/or for unmonitored learning. The machine learning algorithm can be designed for example for deep learning and/or for reinforcement learning and/or for Marginal Space Learning. In particular in the case of monitored learning, a category of functions can be used which is based for example on decision trees, a Random Forest, a logistical regression, a Support Vector Machine, an artificial neural network, a kernel method, Bayes classifiers or the like or combinations thereof. Possible implementations of the machine learning algorithm can use for example artificial intelligence. Optimization methods known to a person skilled in the art can be used for optimization. Calculations, in particular during optimization, can be carried out for example via a processor system. The processor system can have for example one or more graphic processor(s).

The examination protocol can in particular have at least one examination protocol parameter which is selected from the group which comprises an acquisition parameter, a reconstruction parameter, a contrast medium parameter and combinations thereof. An examination protocol parameter can in particular be an acquisition parameter. An acquisition parameter can relate for example to a tube voltage, a tube current, a rotation time, a spiral pitch, one or more trigger instant(s) for a tube current modulation in the cardiac cycle or the like or combinations thereof. An examination protocol parameter can in particular be a reconstruction parameter. A reconstruction parameter can relate for example to a convolution kernel, a convolution algorithm, a slice thickness, a slice increment or the like or combinations thereof. An examination protocol parameter can in particular be a contrast medium parameter. A contrast medium parameter can relate for example to a quantity of contrast medium, a flow rate or the like or combinations thereof.

The status parameters of the medical imaging examination can in particular be patient parameters and/or examination parameters. A status parameters can in particular be a patient parameter, which relates for example to one embodiment or a plurality of embodiments of a condition of the patient to be examined with the examination protocol. The embodiments of the condition of the patient can be in particular demographic, physiological and/or ethnic embodiments.

A patient parameter can be for example a heart rate, a heart rate variability, a size or an attenuation of the X-ray radiation in particular regions of the body of the patient and/or in particular projection directions, an age, a gender, a weight, a height, a Body Mass Index, laboratory values, for example a creatinine value, a density or a concentration of a material in the body of the patient or a variable derived therefrom, a willingness to cooperate, a history, an anamnesis or the like or combinations thereof. For example, a patient parameter, which relates to the willingness to cooperate of the patient, can indicate that the patient is not cooperative. For example, a patient parameter, which relates to the anamnesis of the patient, can indicate that the patient recently had a stroke.

A status parameter can be in particular an examination parameter which relates for example to one or more aspect(s) of the medical imaging examination and/or a clinical process incorporating the medical imaging examination. An examination parameter can relate for example to a referring physician, who referred the patient for medical imaging examination, a user, who is carrying out the medical imaging examination, an indication or the like or combinations thereof.

At least one embodiment of the invention also relates to a data processing unit for determining classification data for an adaption of an examination protocol based on a basic examination protocol of a medical imaging examination as a function of status parameters of the medical imaging examination, comprising:

a training data set supply unit designed for supplying a set of training data sets, wherein every training data set in each case has a status parameter data set with values of the status parameters of the medical imaging examination and an item of adaption information associated with the status parameter data set, wherein the adaption information relates to an in particular manual adaption of the examination protocol, for example by one or more user(s), based on the basic examination protocol of the medical imaging examination, in particular as a function of the status parameters; and

a classification data determining unit designed for determining the classification data based on a machine learning algorithm and the set of training data sets.

In an embodiment, the data processing unit can be designed for determining classification data for carrying out a method for determining classification data according to one or more of the embodiment(s) disclosed in this application.

At least one embodiment of the invention also relates to a method of using classification data, which has been determined according to a method for determining classification data according to one or more of the embodiment(s) disclosed in this application, for optimizing an examination protocol adaption algorithm, which is designed in particular for automatic adaption of an examination protocol based on a basic examination protocol of a medical imaging examination as a function of status parameters of the medical imaging examination. Optimizing an examination protocol adaption algorithm can in particular be taken to mean training of the examination protocol adaption algorithm.

In particular, the examination protocol adaption algorithm can be automatically, in particular continuously, optimized based on the classification data. Alternatively or additionally, a proposal for optimizing an examination protocol adaption algorithm can be generated based on the classification data, which proposal can be adopted by a user.

In particular, training data sets can be supplied which represent the user behavior, for example in which status parameters particular changes to the examination protocol are made by the user. For this purpose, adaptions of examination protocols, in particular changes to examination protocol parameters of the examination protocol relative to the basic examination protocol, together with the status parameters for a large number of medical imaging examinations, in which in particular the same basic examination protocol is used, are logged.

The adaption information can for example a changed value of the basic examination protocol parameter or a change to the changed value of the basic examination protocol parameter relative to the basic examination protocol. The change can be indicated for example absolutely or relatively. The adaption information can indicate, for example, a sign of the change, in other words, a decrease or an increase or a remaining constant of a value. Furthermore, the adaption information can indicate, for example, an allocation of the change to a pre-defined category of changes, in particular a strong or weak change.

Dependencies can be identified therefrom and/or rules derived therefrom using machine learning. Existing automatisms can then be automatically or semi-automatically configured thereby for adaptions of examination protocols. In addition to determining significant dependencies, in this way it can also be determined how consistently this decision is made by the users and whether an automation would achieve the same behavior without manual intervention.

At least one embodiment of the invention also relates to a method of using classification data, which has been determined according to a method for determining classification data according to one or more of the embodiment(s) disclosed in this application, for optimizing a basic examination protocol database which has a plurality of basic examination protocols,

wherein based on the classification data, at least one further basic examination protocol for a scan is determined in the basic examination protocol database and/or

wherein the basic examination protocols of the plurality of basic examination protocols are classified based on the classification data.

In particular, based on the classification data, at least one further basic examination protocol, which was not previously included in the basic examination protocol database, can be determined and/or incorporated in the basic examination protocol database. In particular, the further basic examination protocol can be determined based on the classification data and on a method for adaption of an examination protocol according to one of the embodiments described in this application.

In particular if an automatic adaption of examination protocols is not envisaged, the classification data enables optimization of the permanently stored basic examination protocols by proposing expedient classes of basic examination protocols, for example based on the classification data. The inventive solution therefore enables a simplification of implementation and configuration of medical imaging examinations by the examination protocol adaption algorithms and/or the basic examination protocol databases being in particular automatically adapted by the user to the actual use with the aid of machine learning. This reduces the need for training, for example following a software update, by which new functionality is available and which would not be known to the user without a corresponding indication.

At least one embodiment of the invention also relates to a method for adaption of an examination protocol of a medical imaging examination, wherein the method comprises:

selecting a basic examination protocol of the medical imaging examination;

supplying a data structure in which values of status parameters of a status parameter data set can be stored and changed; and

adaption of an examination protocol based on the basic examination protocol as a function of the status parameters of the status parameter data set.

The examination protocol can in particular be automatically adapted. The examination protocol can in particular be adapted by way of an examination protocol adaption algorithm, which has been optimized for example using classification data which has been determined according to a method for determining classification data according to one or more of the embodiment(s) disclosed in this application. In particular, firstly at least one value of a status parameter of the status parameter data set can be modified in the data structure and then the examination protocol can be adapted based on the basic examination protocol as a function of the at least one changed value of the status parameter of the status parameter data set.

One embodiment of the invention provides

that a user interface is displayed which has a status parameters input element,

that a user input, which relates to the status parameters, is acquired by way of the status parameter input element,

that the value of the status parameter is changed based on the user input, which relates to the status parameter.

One embodiment of the invention provides

that the user interface has a basic examination protocol input element,

that a user input, which relates to the basic examination protocol, is acquired by way of the basic examination protocol input element,

that the basic examination protocol is selected based on the user input, which relates to the basic examination protocol.

One embodiment of the invention provides

that the user interface has an examination protocol output element,

that a value of an examination protocol parameter of the adapted examination protocol and/or a value of the examination protocol parameter of the basic examination protocol are displayed by way of the examination protocol output element.

At least one embodiment of the invention also relates to a data processing unit for adaption of an examination protocol of a medical imaging examination, wherein the data processing unit comprises:

selection unit designed for selecting a basic examination protocol of the medical imaging examination;

data structure supply unit designed for supplying a data structure in which values of status parameters of a status parameter data set can be stored and changed; and

adaption unit designed for adaption of an examination protocol based on the basic examination protocol as a function of the status parameters of the status parameter data set.

In particular, the data processing unit can be designed for adaption of an examination protocol for carrying out a method for adaption of an examination protocol according to one or more of the embodiment(s) disclosed in this application. Optionally, the data processing unit can have a changing unit for adaption of an examination protocol designed for changing at least one value of a status parameter of the status parameter data set in the data structure.

At least one embodiment of the invention also relates to a non-transitory computer program product having a computer program, which can be loaded directly into a storage device of a computer, having program segments in order to carry out all steps of a method according to one or more of the embodiment(s) disclosed in this application when the computer program is executed in the computer.

At least one embodiment of the invention also relates to a non-transitory computer-readable medium on which program segments that can be read and executed by a computer are stored in order to carry out all steps of a method according to one of the embodiment(s) disclosed in this application when the program segments are executed by the computer.

The data structure, in which values of status parameters of a status parameter data set can be stored and changed can be designed in the form of a virtual patient. It is thereby possible for example to configure a virtual patient and in particular to check and/or visualize the input status parameters for plausibility. Clinical settings, in particular examination protocol adaption algorithms, can therefore be validated and configured on such virtual patients outside of a real clinical case. The status parameters of the virtual patient can be displayed graphically and/or using the medical jargon in a manner that is easy and straightforward to identify.

In particular, in this way status parameters of reference patients can be visualized consistently and comprehensively for the user. Furthermore, status parameter data sets, supplied by the manufacturer as a default value, which correspond to the most common types of patient, can thereby be provided to the user in the form of virtual patients for selection and/or further adaptation. The inventive solution makes it possible in particular for the user, for example via the user interface, to compile status parameter data sets individually and for example adapt them to patients to be examined.

For the configuration of the status parameter data set, in particular in the form of the virtual patient, the user can be provided via the user interface with a pool of status parameters, which can then be selected and adapted for example by user interactions.

A status parameter data set, which represents a virtual patient, can also be created on the basis of real patient data. For example, the status parameters of patients, which represent a frequently treated patient group very well, can be stored in the form of a virtual patient in the computer of the medical imaging device. Even more realistic and complete information can be supplied in this way. A change to clinical settings can then be validated on a representative virtual patient. An application of this kind can be of interest inter alia to hospitals which have specialized in particular types of patient. A further application would be conceivable for example in demonstration pre-settings since in this way a complex algorithm can be tested and explained using an easily imaginable, virtual patient. Incorrect inputs and limit values, which could lead to an increased risk for the real patient, can therefore also be tried out and demonstrated without exposing the real patient themselves to the increased risk.

Due to the configuration of a status parameter data set in the form of a virtual patient, the inventive solution enables clear representation and handling of the otherwise very technical parameters, and therewith a plausibility check of the input data. In addition to a better understanding and a higher identification with the input data, the quality also increases since incorrect inputs can be minimized. A further advantage is the possibility of calling the virtual patient by their usual personal name. This also enhances identification and better communication since it is not the technical parameters that are discussed but the various virtual patients.

The data processing unit for determining classification data and/or the data processing unit for adaption of an examination protocol and/or one or more component(s) thereof can be formed by a data processing system. The data processing system can have for example one or more component(s) in the form of hardware and/or one or more component(s) in the form of software.

The data processing system can for example be formed at least partially by a cloud computing system. The data processing system can be and/or have for example a cloud computing system, a computer network, a computer, a tablet computer, a smartphone or the like or a combination thereof. The hardware can cooperate for example with software and/or be configured by way of software. The software can be executed for example by way of the hardware. The hardware can be for example a storage system, an FPGA system (Field-programmable gate array), an ASIC system (Application-specific integrated circuit), a microcontroller system, a processor system and combinations thereof. The processor system can have for example a microprocessor and/or a plurality of cooperating microprocessors.

In particular, one component of the data processing unit according to one of the embodiments, which are disclosed in this application, which is designed to carry out a given step of a method according to one of the embodiments, which are disclosed in this application, can be implemented in the form of hardware, which is configured for carrying out the given step and/or which is configured for carrying out a computer-readable instruction in such a way that the hardware can be configured by way of the computer-readable instruction for carrying out the given step. In particular, the system can have a storage area, for example in the form of a computer-readable medium, in which computer-readable instructions, for example in the form of a computer program, are stored.

Data can be transferred between components of the data processing system for example in each case via a suitable data transfer interface. The data transfer interface for data transfer to and/or from a component of the data processing system can be implemented at least partially in the form of software and/or at least partially in the form of hardware. The data transfer interface can be designed for example for storing data in and/or for loading data from a sector of the storage system, it being possible to access one or more component(s) of the data processing system on this sector of the storage system.

In particular, data, which relates for example to a medical image, examination protocol parameters, status parameters or classification data, can be supplied by loading the data, for example from a sector of a storage system, and/or generated, for example via a medical imaging device.

The computer program can be loaded in the storage system of the data processing system and be executed by the processor system of the data processing system. The data processing system can be designed for example by way of the computer program in such a way that the data processing system can carry out the steps of a method according to one of the embodiments which are disclosed in this application, when the computer program is executed by the data processing system.

The computer program product can be for example the computer program or comprise at least one additional component in addition to the computer program. The at least one additional component of the computer program product can be designed as hardware and/or as software. The computer program product can have for example a storage medium on which at least some of the computer program product is stored, and/or a key for authentication of a user of the computer program product, in particular in the form of a dongle.

The computer program product and/or the computer program can have for example a cloud application program, which is designed for distributing program segments of the computer program among various processing units, in particular various computers, of a cloud computing system, wherein each of the processing units is designed for carrying out one or more program segment(s) of the computer program. For example the computer program product according to one of the embodiments which are disclosed in this application, and/or the computer program according to one of the embodiments which are disclosed in this application can be stored on the computer-readable medium. The computer-readable medium can be for example a memory stick, a hard disk or another data carrier which can in particular be detachably connected to the data processing system or be permanently integrated in the data processing system. The computer-readable medium can form for example a sector of the storage system of the data processing system.

The medical imaging examination can in particular be a medical imaging examination via a medical imaging device. The medical imaging device can be selected for example from the imaging modalities group which comprises an X-ray apparatus, a C-arm X-ray apparatus, a computed tomography scanner (CT scanner), a molecular imaging scanner (MI scanner), a Single Photon Emission Computed Tomography scanner (SPECT scanner), a Positron Emission Tomography scanner (PET scanner), a magnetic resonance tomography scanner (MR scanner) and combinations thereof, in particular a PET-CT scanner and a PET-MR scanner. The medical imaging device can also have a combination of an imaging modality, which is selected for example from the imaging modalities group, and an irradiation modality. The irradiation modality can have for example an irradiation unit for therapeutic irradiation. The medical imaging device can have for example a contrast medium injector.

Without limiting the general inventive idea, in some of the embodiments a computed tomography scanner is cited by way of example for a medical imaging device.

According to one embodiment of the invention, the medical imaging device has an acquisition unit which is designed for acquisition of the acquisition data. In particular, the acquisition unit can have a radiation source and a radiation detector. One embodiment of the invention provides that the radiation source is designed for emission and/or for excitation of radiation, in particular an electromagnetic radiation and/or that the radiation detector is designed for detection of the radiation, in particular the electromagnetic radiation. The radiation can pass for example from the radiation source to a region to be imaged and/or, after an interaction with the region to be imaged, to the radiation detector. During the interaction with the region to be imaged, the radiation is modified and thereby becomes a carrier of information relating to the region to be imaged. During the interaction of the radiation with the detector, this information is acquired in the form of acquisition data.

In particular with a computed tomography scanner and with a C-arm X-ray apparatus, the acquisition data can be projection data, the acquisition unit a projection data acquisition unit, the radiation source an X-ray source, the radiation detector an X-ray detector. The X-ray detector can in particular be a quantum-counting and/or energy-resolving X-ray detector.

Within the context of embodiments of the invention, features which are described in relation to different embodiments of the invention and/or different claims categories (method, use, device, system, arrangement, etc.), can be combined to form further embodiments of the invention. For example, a claim which relates to a device can also be developed with features, which are described or claimed in connection with a method, and vice versa. Functional features of a method can be implemented by appropriately designed concrete components. In addition to the embodiments of the invention explicitly described in this application, a wide variety of further embodiments of the invention is conceivable which a person skilled in the art can arrive at without departing from the scope of the invention insofar as it is specified by the claims.

Use of the indefinite article “a” or “an” does not preclude the relevant feature from also being present multiple times. Use of the expression “to have” does not preclude the terms linked by means of the expression “to have” from being identical. For example, the medical imaging device has the medical imaging device. Use of the expression “unit” does not preclude the article, to which the expression “unit” refers, from having a plurality of components which are spatially separated from each other. In the context of the present application, the expression “based on” can in particular be understood within the meaning of the expression “using”. In particular wording, which is generated as a result of a first feature based on a second feature (alternatively: ascertained, determined, etc.), does not preclude the first feature from being generated on the basis of a third feature (alternatively: ascertained, determined, etc.).

The invention will be illustrated below using exemplary embodiments and with reference to the accompanying figures. The illustration in the figures is schematic, highly simplified and not necessarily to scale.

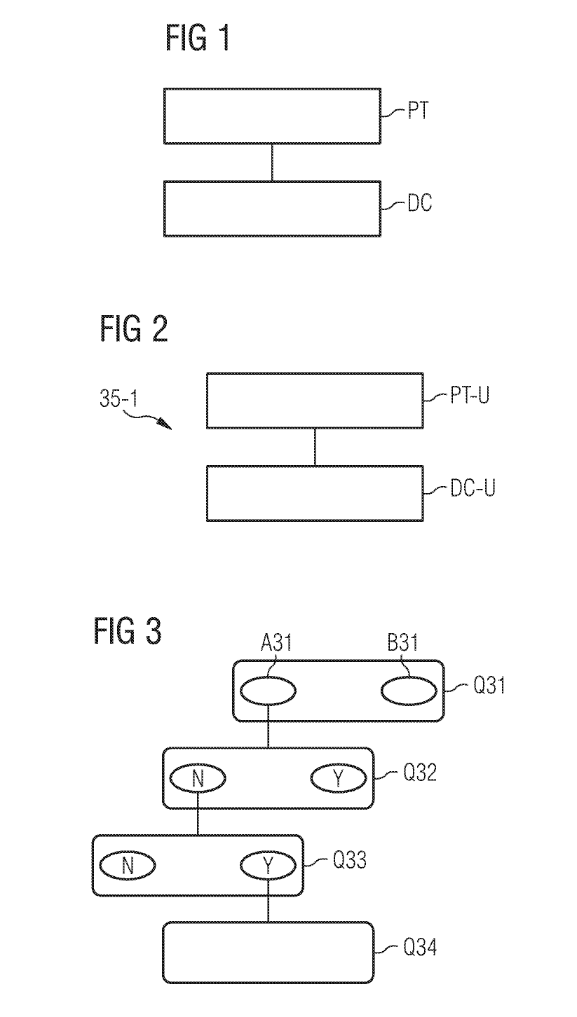

FIG. 1 shows a flowchart of a method for determining classification data for adaption of an examination protocol based on a basic examination protocol of a medical imaging examination as a function of status parameters of the medical imaging examination, wherein the method comprises the following steps:

supplying PT a set of training data sets, wherein every training data set in each case has a status parameter data set with values of the status parameters of the medical imaging examination and an item of adaption information associated with the status parameter data set, wherein the adaption information relates to an adaption of the examination protocol based on the basic examination protocol of the medical imaging examination, and

determining DC the classification data based on a machine learning algorithm and the set of training data sets.

FIG. 2 shows a schematic illustration of a data processing unit 35-1 for determining classification data for adaption of an examination protocol based on a basic examination protocol of a medical imaging examination as a function of status parameters of the medical imaging examination, having:

a training data set supply unit PT-U designed for supplying PT a set of training data sets, wherein every training data set in each case has a status parameter data set with values of the status parameters of the medical imaging examination and an item of adaption information associated with the status parameter data set, wherein the adaption information relates to an adaption of the examination protocol based on the basic examination protocol of the medical imaging examination,

a classification data determining unit DC-U designed for determining DC the classification data based on a machine learning algorithm and the set of training data sets.

FIGS. 3-5 show examples of learned decision trees. A decision is also possible when only one subset of status parameters considered overall in the classification data is available for querying. Decisions for particular partial embodiments of the examination protocol can then be supported by determining separate branches in whose nodes only the available status parameters are queried.

The decision tree, which is shown in FIG. 3, relates to the selection of an acquisition method as a function of age, heart rate and the heart rate variability of the patient 13. In node Q31, a spiral method with high pitch is proposed and it is queried which of the age groups, Adult A31 or Child B31, patient 13 is associated with. If the patient 13 is associated with age group Adult A31, a triggered sequence is proposed in node Q32 and it is queried whether the heart rate exceeds Y a particular threshold value, for example 65 beats per minute, or not N. If the heart rate does not exceed the particular threshold value N, a spiral method with high pitch is proposed in node Q33 and it is queried whether the heart rate variability exceeds Y a particular threshold value, for example 7 beats per minute, or not N. If the heart rate variability exceeds Y the particular threshold value, a triggered sequence is proposed in node Q34.

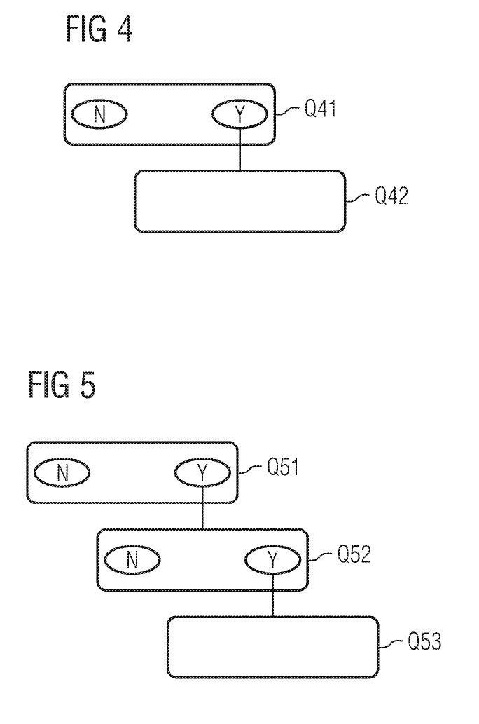

The decision tree, which is shown in FIG. 4, relates to a modification of the exposure to radiation as a function of the Agatston score (Calcium score). A default value for the exposure to radiation is proposed in the node Q41 and it is queried whether the Agatston score exceeds Y a particular threshold value, for example 400, or not N. If the Agatston score exceeds Y the particular threshold value, an exposure to radiation is proposed in node Q42 which is higher by a particular factor, for example 1.5, than in the default value for the exposure to radiation.

The decision tree, which is shown in FIG. 5, relates to the adaption of a reconstruction algorithm as a function of a mean patient diameter. A first kernel, for example kernel Br40, is proposed in node Q51 and it is queried whether the mean patient diameter exceeds Y a particular first threshold value, for example 38 cm, or not N. A second kernel, for example kernel Br36, is proposed in node Q52 and it is queried whether the mean patient diameter exceeds Y a particular second threshold value, for example 50 cm, or not N. A third kernel, for example kernel Br32, is proposed in node Q53.

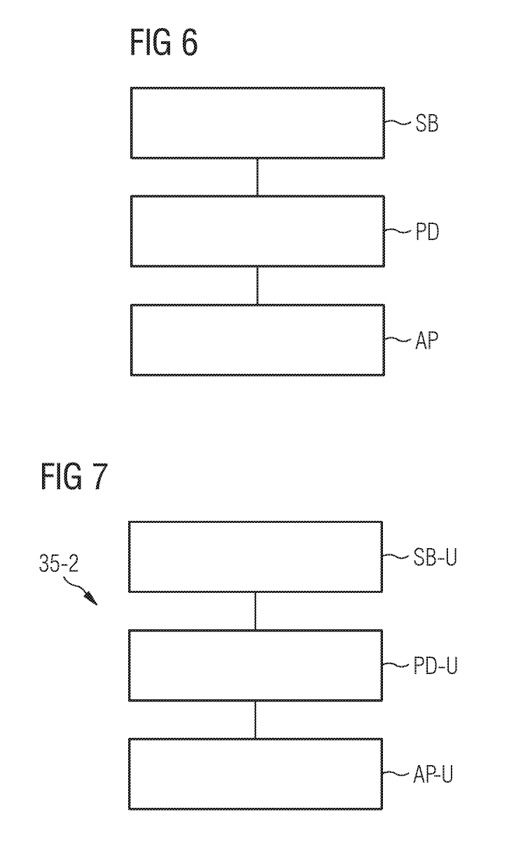

FIG. 6 shows a flowchart of a method for adaption of an examination protocol of a medical imaging examination, wherein the method comprises the following steps:

selecting SB a basic examination protocol of the medical imaging examination,

supplying PD a data structure in which values of status parameters of a status parameter data set can be stored and changed,

changing CV at least one value of a status parameter of the status parameter set in the data structure,

adaption AP of an examination protocol based on the basic examination protocol as a function of the status parameters of the status parameter data set.

FIG. 7 shows a schematic illustration of a data processing unit for adaption of an examination protocol of a medical imaging examination, wherein the method comprises the following steps:

selection unit SB-U designed for selecting SB a basic examination protocol of the medical imaging examination,

data structure supply unit PD-U designed for supplying PD a data structure, in which values of status parameters of a status parameter data set can be stored and changed,

adaption unit AP-U designed for adaption AP of an examination protocol based on the basic examination protocol as a function of the status parameters of the status parameter data set.

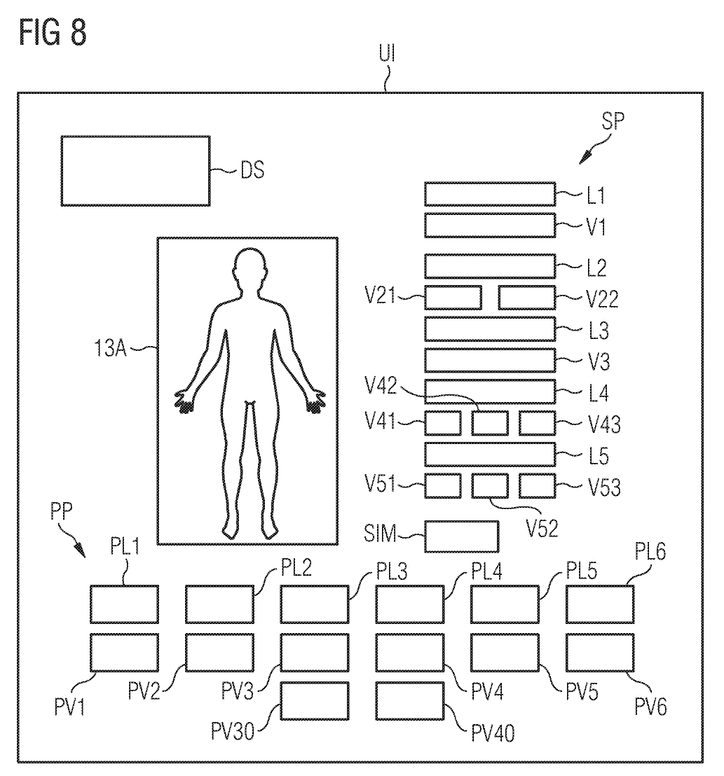

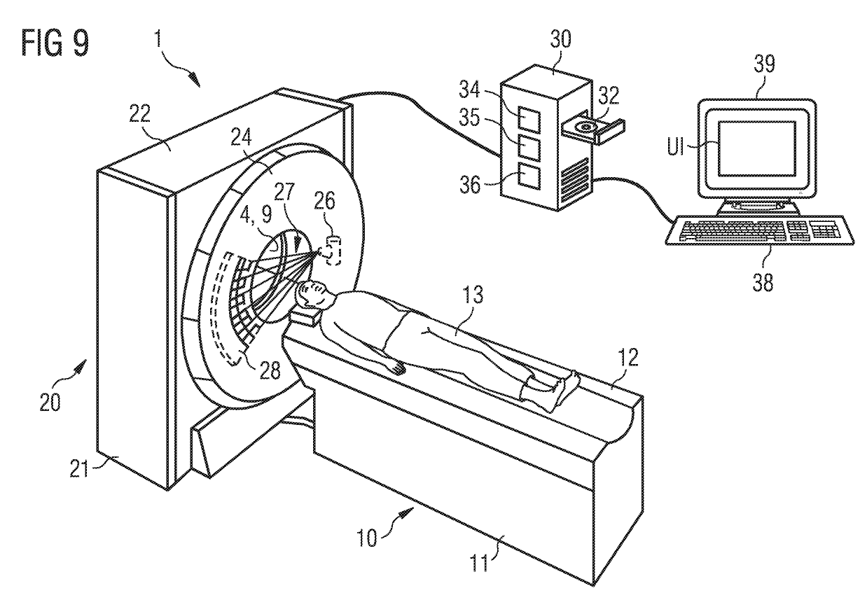

FIG. 8 shows a user interface UI for adaption of an examination protocol. The user interface UI has the basic examination protocol input element V1 in the form of a dropdown list for selecting a basic examination protocol. The label L1 “Contrast medium protocol” is associated with the basic examination protocol input element V1, and this points to the function of the basic examination protocol input element V1.

The user interface UI has the following status parameter input elements in the status parameter input area SP: gender of the patient: buttons V21 for male and V22 for female, weight of the patient: text input field V3, age group of the patient: for example buttons V41 for 18-30 years, V42 for 30-65 years, V43 for older than 65 years, renal function efficiency: buttons V51 for normal, V52 for reduced, V53 for severely damaged. The labels L2 “gender”, L3 “weight”, L4 “age” and L5 “renal function efficiency” respectively are associated with the status parameter input elements, and these point to the function of the corresponding status parameter input elements. In addition, the status parameter input elements themselves can each have a label which describes the value, which can be input with the status parameter input elements, in more detail.

The user interface UI has the following examination protocol output elements in the form of text display fields in the examination protocol output area PP, which relate to contrast medium parameters in each case: PV1 for the contrast medium name: Ultravist, PV2 for the iodine concentration in mg/ml: 370, PV3 for the flow in ml/s: 3.3, PV4 for the volume in ml: 80, PV5 for the duration in seconds: 24, PV6 for the contrast medium ratio in percent: 100. The labels PL1 “contrast medium name”, PL2 “iodine concentration in mg/ml”, PL3 “flow in ml/s”, PL4 “volume in ml”, PL5 “duration in seconds”, PL6 “contrast medium ratio in percent” respectively are associated with the examination protocol output elements, and these point to the function of the corresponding examination protocol output elements.

In addition, the user interface UI has the examination protocol output elements PV30 and PV40 in the form of text display fields, wherein via PV30 a value for the flow is displayed in ml/s according to the basic examination protocol and via PV40 a value for the volume is displayed in s according to the basic examination protocol. Examination protocol output elements can also be provided in which a relative or absolute change in the value of the basic examination protocol parameter relative to the basic examination protocol is displayed.

Without limiting the general inventive idea, contrast medium parameters are shown by way of example for the examination protocol parameters. Other examination protocol parameters, for example acquisition parameters and/or acquisition parameters, can also be adapted alternatively or additionally to the contrast medium parameters.

An avatar of the selected virtual patient for example can be shown in the image display field 13A. Using the button DS, the default values according to the unchanged status parameter data set can be loaded for the selected virtual patient. Using the button SIM, automatic determination of the values can be started for the examination protocol parameters based on the current status parameters and the selected basic examination protocol. It can also be provided that the examination protocol parameters are updated in real time as a function of the changed status parameters.

Without limiting the general inventive idea, a computed tomography scanner is shown by way of example for the medical imaging device 1. The medical imaging device 1 has the gantry 20, the tunnel-like opening 9, the patient support device 10 and the control device 30. The gantry 20 has the stationary support frame 21, the tilting frame 22 and the rotor 24. The tilting frame 22 is arranged on the stationary support frame 21 via a tilt bearing device so as to be tiltable relative to the stationary support frame 21 about a tilt axis. The rotor 24 is arranged on the tilting frame 22 via a pivot bearing device so as to be rotatable about an axis of rotation relative to the tilting frame 22.

The patient 13 can be introduced into the tunnel-like opening 9. The acquisition region 4 is situated in the tunnel-like opening 9. A region to be imaged of the patient 13 can be positioned in the acquisition region 4 in such a way that the radiation 27 can pass from the radiation source 26 to the region to be imaged and, after an interaction with the region to be imaged, to the radiation detector 28. The patient support device 10 has the support base 11 and the support panel 12 for supporting the patient 13. The support panel 12 is arranged on the support base 11 so as to be moveable relative to the support base 11 in such a way that the support panel 12 can be introduced in a longitudinal direction of the support panel 12, in particular along the system axis AR, into the acquisition region 4.

The medical imaging device 1 is designed for acquisition of acquisition data based on electromagnetic radiation 27. The medical imaging device 1 has an acquisition unit. The acquisition unit is a projection data acquisition unit having the radiation source 26, for example an X-ray source, and the detector 28, for example an X-ray detector, in particular an energy-resolving X-ray detector. The radiation source 26 is arranged on the rotor 24 and designed for emission of radiation 27, for example X-ray radiation, with radiation quanta 27. The detector 28 is arranged on the rotor 24 and designed for detection of the radiation quanta 27. The radiation quanta 27 can pass from the radiation source 26 to the region to be imaged of the patient 13 and, after an interaction with the region to be imaged, strikes the detector 28. In this way, acquisition data of the region to be imaged can be acquired in the form of projection data via the acquisition unit.

The control device 30 is designed for receiving the acquisition data acquired by the acquisition unit. The control device 30 is designed for controlling the medical imaging device 1. The control device 30 has the data processing unit 35, the computer-readable medium 32 and the processor system 36. The control device 30, in particular the data processing unit 35, is formed by a data processing system which has a computer. The data processing unit 35 can be the data processing unit 35-1 for determining classification data and/or the data processing unit 35-2 for adaption of an examination protocol. The control device 30 has the image reconstruction device 34. A medical image data set can be reconstructed via the image reconstruction device 34 on the basis of the acquisition data.

The medical imaging device 1 has an input device 38 and an output device 39 auf, which are each connected to the control device 30. The input device 38 is designed for inputting control information, for example image reconstruction parameters, examination parameters or the like. The output device 39 is designed in particular for outputting control information, images and/or acoustic signals. The output device 39 can in particular be a screen with which the user interface UI can be displayed.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for determining classification data for an adaption of an examination protocol based on a basic examination protocol of a medical imaging examination as a function of status parameters of the medical imaging examination, the method comprising:
supplying a set of training data sets, each training data set of the set of training data sets including a status parameter data set with values of the status parameters of the medical imaging examination and an item of adaption information associated with the status parameter data set, wherein the adaption information relates to an adaption of the examination protocol based on the basic examination protocol of the medical imaging examination; and
determining the classification data based on a machine learning algorithm and the set of training data sets.

2. The method of claim 1, wherein at least one of
the classification data forms a decision tree, and
the machine learning algorithm is based on recursive partitioning.

3. The method of claim 1, wherein the examination protocol includes at least one examination protocol parameter, selected from a group comprising an acquisition parameter, a reconstruction parameter, a contrast medium parameter and combinations of at least two of the acquisition parameter, the reconstruction parameter, and the contrast medium parameter.

4. The method of claim 1, wherein the status parameters of the medical imaging examination are at least one of patient parameters and examination parameters.

5. A data processing unit for determining classification data for an adaption of an examination protocol based on a basic examination protocol of a medical imaging examination as a function of status parameters of the medical imaging examination, comprising:
a training data set supply unit designed to supply a set of training data sets, each training data set of the set of training data sets including a status parameter data set with values of the status parameters of the medical imaging examination and an item of adaption information associated with the status parameter data set, wherein the adaption information relates to an adaption of the examination protocol based on the basic examination protocol of the medical imaging examination; and
a classification data determining unit designed to determine the classification data based on a machine learning algorithm and the set of training data sets.

6. A method, comprising:
supplying a set of training data sets, each training data set of the set of training data sets including a status parameter data set with values of status parameters of a medical imaging examination and an item of adaption information associated with the status parameter data set, wherein the adaption information relates to an adaption of the examination protocol based on a basic examination protocol of the medical imaging examination;
determining classification data based on a machine learning algorithm and the set of training data sets; and
using the classification data determined to optimize an examination protocol adaption algorithm, designed for adaption of the examination protocol based on the basic examination protocol of the medical imaging examination as a function of status parameters of the medical imaging examination.

7. A method for optimizing a basic examination protocol database, including a plurality of basic examination protocols, the method comprising:
determining the classification data based upon the method of claim 1; and at least one of
determining, based on the classification data determined, at least one further basic examination protocol for a scan in the basic examination protocol database, and
classifying the basic examination protocols of the plurality of basic examination protocols based on the classification data determined.

8. A method for adaption of an examination protocol of a medical imaging examination, the method comprising:
selecting a basic examination protocol of the medical imaging examination;
supplying a data structure, to store changeable values of status parameters of a status parameter data set; and
adapting the examination protocol based on the basic examination protocol selected as a function of the status parameters of the status parameter data set.

9. A method for adaption of an examination protocol of a medical imaging examination, the method comprising:
selecting a basic examination protocol of the medical imaging examination;
supplying a data structure, to store changeable values of status parameters of a status parameter data set; and
adapting the examination protocol based on the basic examination protocol selected as a function of the status parameters of the status parameter data set,
wherein the adapting of the examination protocol takes place using an examination protocol adaption algorithm, optimized using classification data determined according to the method of claim 1.

10. The method of claim 8,
wherein a user interface is displayed, including a status parameter input element,
wherein a user input, relating to the status parameter, is acquired using the status parameter input element, and
wherein the value of the status parameter is changed based on the user input, relating to the status parameter.

11. The method of claim 10,
wherein the user interface includes a basic examination protocol input element,
wherein a user input, relating to the basic examination protocol, is acquired using the basic examination protocol input element, and
wherein the basic examination protocol is selected based on the user input, relating to the basic examination protocol.

12. The method of claim 10,
wherein the user interface includes an examination protocol output element, and
wherein at least one of a value of an examination protocol parameter of the examination protocol adapted and a value of the examination protocol parameter of the basic examination protocol is displayed using the examination protocol output element.

13. A data processing unit for adaption of an examination protocol of a medical imaging examination, the data processing unit comprising:
selection unit to select a basic examination protocol of the medical imaging examination;
data structure supply unit to supply a data structure in which values of status parameters of a status parameter data set can be stored and changed; and
adaption unit to adapt of an examination protocol based on the basic examination protocol as a function of the status parameters of the status parameter data set.

14. A non-transitory computer program product storing a computer program, directly loadable into a storage device of a computer, including program segments to carry out the method of claim 1 when the program segments are executed by the computer.

15. A non-transitory computer-readable medium storing program segments, readable and executable by a computer, to carry out the method of claim 1 when the program segments are executed by the computer.

16. The method of claim 2, wherein the examination protocol includes at least one examination protocol parameter, selected from a group comprising an acquisition parameter, a reconstruction parameter, a contrast medium parameter and combinations of at least two of the acquisition parameter, the reconstruction parameter, and the contrast medium parameter.

17. The method of claim 2, wherein the status parameters of the medical imaging examination are at least one of patient parameters and examination parameters.

18. The method of claim 8,
wherein a user interface is displayed, including a status parameter input element,
wherein a user input, relating to the status parameter, is acquired using the status parameter input element, and
wherein the value of the status parameter is changed based on the user input, relating to the status parameter.

19. The method of claim 18,
wherein the user interface includes a basic examination protocol input element,
wherein a user input, relating to the basic examination protocol, is acquired using the basic examination protocol input element, and
wherein the basic examination protocol is selected based on the user input, relating to the basic examination protocol.

20. The method of claim 18,
wherein the user interface includes an examination protocol output element, and
wherein at least one of a value of an examination protocol parameter of the examination protocol adapted and a value of the examination protocol parameter of the basic examination protocol is displayed using the examination protocol output element.
